(19) United States
(12) Patent Application Publication
(10) Pub. No.: US 2020/0352854 A1
(43) Pub. Date: Nov. 12, 2020

(54) DEVICES AND METHODS FOR PREVENTING AND INHIBITING PERIODONTAL DISEASE

(71) Applicant: Fixed Focus LLC, River Vale, NJ (US)

(72) Inventors: Steven BLECHMAN, Valley Cottage, NY (US); Stephen A. Roth, Gladwyne, PA (US); Peter Kash, River Vale, NJ (US)

(73) Assignee: Fixed Focus LLC, River Vale, NJ (US)

(21) Appl. No.: 16/405,419

(22) Filed: May 7, 2019

(51) Int. Cl. A61K 9/00; A61C 19/06; A61K 33/16; A61K 9/70; A61Q 11/00

(52) U.S. Cl. CPC A61K 9/0063; A61C 19/06; A61K 6/0067; A61K 9/7007; A61Q 11/00; A61K 33/16

(57) ABSTRACT

A dental tool is provided which is capable of delivering an oligosaccharide containing composition to an oral biofilm, which can prevent and inhibit periodontal disease and in particular to devices and methods which mechanically and chemically disrupt biofilms formed by S. mutans.

### BACKGROUND OF THE INVENTION

### Field of the Invention

This invention relates to devices and methods for preventing and inhibiting periodontal disease and in particular to devices and methods which mechanically and chemically disrupt biofilms formed by S. mutans.

### Discussion of the Background

Periodontal or gum disease is a pathological inflammatory condition of the gum and bone support (periodontal tissues) surrounding the teeth and is commonly presented in the form of gingivitis, an inflammation of the gum at the necks of the teeth and periodontitis, inflammation affecting the bone and the tissue of the teeth. Gingivitis is characterized by redness of the gum margins, swelling and bleeding on brushing.

Gingivitis occurs in both chronic and acute forms. Acute gingivitis is usually associated with specific infections, micro-organisms, or trauma. Chronic inflammation of the gum tissue surrounding the teeth is associated with the bacterial biofilm (plaque) that covers the teeth and gums. Gingivitis was once seen as the first stage in a chronic degenerative process which resulted in the loss of both gum and bone tissue surrounding the teeth. It is now recognized that gingivitis can be reversed by effective personal oral hygiene practices.

### Periodontitis

When periodontal disease affects the bone and supporting tissue, it is termed periodontitis and is characterized by the formation of pockets or spaces between the tooth and gums.

This may progress and cause chronic periodontal destruction leading to loosening or loss of teeth. The dynamics of the disease are such that the individual can experience episodes of rapid periodontal disease activity in a relatively short period of time, followed by periods of remission.

Though the majority of adults are affected by gingivitis, gingivitis fortunately does not always develop into periodontal disease. Progression of gum disease is influenced by a number of factors which include oral hygiene and genetic predisposition. One of the challenges for early detection of periodontal disease is its “silent” nature—the disease does not cause pain and can progress unnoticed. In its early stages, bleeding gums during toothbrushing may be the only sign; as the disease advances and the gums deteriorate, the bleeding may stop and there may be no further obvious sign until the teeth start to feel loose. In most cases, periodontal disease responds to treatment and although the destruction is largely irreversible its progression can be halted.

### Factors Affecting Periodontal Disease

The rate of progression of periodontal disease in an individual is dependent on the virulence (or strength of attack) of the bacterial plaque and on the efficiency of the local and systemic immunoinflammatory responses in the person (host). The overall balance between the bacterial plaque challenge and the body's immunoinflammatory responses is critical to periodontal health. Current research suggests that host responses are influenced by specific environmental and genetic factors which can determine the general susceptibility of the host or the local susceptibility of a site (tooth) within the mouth to periodontal disease. In this regard, it is common for more severe forms of periodontal disease to present in individuals with compromised immune systems, e.g., those with diabetes, HIV infection, leukemia and Down syndrome.

Because periodontal disease is linked to an increased susceptibility to systemic disease (e.g., cardiovascular disease, infective endocarditis, bacterial pneumonia, low birth weight, diabetes) , it is important not only for oral health but also for general health to control periodontal disease.

### Plaque Control for Gingival Health

Plaque control is the most important method of limiting periodontal disease and maintaining gingival health. This must be considered at two levels: what individuals can do for themselves by way of plaque control on a daily basis, and what dentists and hygienists can do to eliminate plaque retention factors in individuals and to advise patients on the most appropriate home care.

### Aids to Plaque Removal

Plaque removal can be aided with the use of: Plaque disclosing agents; dental floss and other interdental cleaning aids; and mouth rinses.

Biofilms form when bacteria such as S. mutans adhere to surfaces in some form of watery environment and begin to excrete a slimy, glue-like substance (polyglucan) some soluble, some insoluble that can stick to all kinds of materials—metals, plastics, soil particles, medical implant materials, biological tissues. Dental plaque is a biofilm that adheres to tooth and other oral surfaces, particularly at the gingival margin, and is implicated in the occurrence of gingivitis, periodontitis, caries and other forms of periodontal disease. Dental plaque is cohesive and highly resistant to removal from teeth and/or oral surfaces. Anaerobic bacteria such as Actinomyces spp. in plaque metabolize sugar to produce acids which dissolve tooth minerals, damaging the enamel and eventually forming dental caries. Saliva can buffer acids produced by bacteria and promote remineralization of the enamel, but extensive plaque can block the saliva from contact with the enamel. Redeposition of minerals in the biofilm forms a hard deposit on the tooth called calculus (or tartar), which becomes a local irritant for the gums, causing gingivitis.

Dani et al. 2016 Oct-Dec; 7(4): 529-534 report an increase colonization of S. mutans in chronic periodontitis subjects both in saliva and sub-gingival plaque samples.

Ahn et al. Infection and Immunity, Sept 2008, 4259-4268 report on the characteristics of biofilm formation of S. mutans in the presence of saliva.

Stoudt et al. U.S. Pat. Nos. 4,340,673 and 4,430,322 discloses certain glucans can modify the biosynthetic route of extra-cellular polysaccharides involved in dental plaque development.

Chen et al. Expert Opin Ther Pat. 2010 May; 20(5): 681-694 reviews the etiology of dental caries and the development of technologies for the prevention and treatment of dental caries.

Ren et al. Antimicrobial Agents and Chemotherapy January 2016 volume 60, no 1 126-135 discloses an investigation to identify novel molecules that target glucosyltransferases to inhibit S. mutans biofilm formation.

Takata et al. Infection and Immunity, Dec. 1985, p 833-843 discloses inhibition of plaque and caries formation by a glucan produced by S. mutans mutant UAB108.

Lemos et al. Microbiology (2013) 159, 436-445 discloses an investigation into the genetics, biochemistry and physiology of the dental pathogen S. mutans, an organism that evolved in close association with the human host, as a novel Gram-positive model organism.

Bowen et al. Caries Res. (2011) Apr; 45(1): 69-86 discuss the biology of S. mutans-derived glucosyltransferases and the role of extracellular matrix formation of cariogenic biofilms.

Osawa et al. J. Dent Res 80(11): 2000-2004, 2001 discloses cariostatic substances showing anti-glucosyltransferase activity and antibacterial activity in cacao bean husk.

Anastassiades U.S. Pat. No. 10,239,962 discloses to hyaluronic acid derivatives, and in particular, derivatives in which the N-acetyl group of hyaluronic acid has been substituted, and methods and uses thereof.

Nemeh et al. U.S. Pat. No. 10,312,598 discloses a method and apparatus for the concurrent treatment of multiple oral diseases and defects while promoting general oral hygiene utilizing direct current electricity and methods for manufacturing the same.

Attstrom et al. U.S. Pat. No. 10,206,928 discloses a composition comprising Delmopinol (or a derivative or salt thereof) is effective in maintaining the oral health of animals, in particular companion animals.

Tsugane et al. U.S. Pat. No. 10,201,493 discloses plant extracts of potherb mustard, Japanese mustard spinach, hot radish and peppergrass belonging to the Brassicaceae family, Japanese angelica tree belonging to the Araliaceae family, and ice plant belonging to the Aizoaceae family has an inhibitory effect on the A. naeslundii biofilm formation induced by acid.

Reed et al. U.S. Pat. No. 10,104,888 discloses biodegradable and biocompatible tannin-chitosan composites. The new composites can be formed into a variety of materials such as hydrogel films, three-dimensional foams, nanoparticles, and liposome coatings. The tannin-chitosan composite materials are stronger and have better mechanical properties than known chitosan materials.

Deisenroth et al. U.S. Pat. No. 10,098,830 discloses polyesters formed from xylitol, polycarboxylic acids (or esters, acid halides or anhydrides thereof) and optionally arginine. The formed polyesters or polyesteramides are active in biofilm inhibition and dissolution to maintain clean teeth.

Avramoff et al. U.S. Pat. No. 9,889,090 discloses an oral delivery device for controlled release of a solid unit dosage form suitable for insertion into a periodontal pocket of a patient, comprising a therapeutically effective amount of an active ingredient selected from: i) at least one anti-inflammatory agent, ii) at least one antibacterial agent, and iii) the combination of at least one anti-inflammatory agent and at least one antibacterial agent.

Mordas et al. U.S. Pat. Nos. 9,591,852 and 9,848,600 discloses a biofilm disruptor comprising at least one unsaturated aliphatic long chain alcohols and/or aldehydes, or combinations of such compounds.

Looper et al. U.S. Pat. No. 9,839,219 discloses compounds, compositions, and methods comprising a polyamine compound are described, which may be used to kill, disperse, treat, or reduce biofilms, or to inhibit or substantially prevent biofilm formation.

Burgess et al. U.S. Pat. No. 9,675,736 reports anti-biofouling compositions including microbial deoxyribonucleases for the disruption of biofilm and prevention of biofilm on surfaces. The invention also relates to the removal of biological material from surfaces.

Tsuchida et al. U.S. Pat. No. 8,343,556 discloses a composition for treating and/or preventing the periodontal disease, which comprises an extract originated from the plant: Sasa (bamboo grass).

Cutler U.S. Pat. No. 5,900,230 discloses treat and prevent of periodontal disease with dental products contain a synergistic mixture of poloxamers, and/or poloxamer congeners, plus xylitol.

Jamas et al. U.S. Pat. Nos. 5,622,939 and U.S. 5,817,643 report neutral, aqueous soluble β-glucans which exert potent and specific immunological effects without stimulating the production of certain cytokines, to preparations containing the novel beta-glucans.

Kohli et al. U.S. Pat. No. 9,888,988 discloses a dental floss comprising a basic amino acid or salt thereof.

Streptococcus mutans is a facultatively anaerobic, Gram-positive coccus (round bacterium) commonly found in the human oral cavity and is a significant contributor to tooth decay.

Porphyromonas gingivalis belongs to the genus Bacteroidetes and is a nonmotile, Gram-negative, rod-shaped, anaerobic, pathogenic bacterium. It is found in the oral cavity, where it is implicated in periodontal disease, as well as in the upper gastrointestinal tract, the respiratory tract and the colon. P. gingivalis infection has been linked to Alzheimer's disease and rheumatoid arthritis.

Some efforts to treat periodontal disease have taken an antibacterial approach. Such effort can be complicated by a significant disruption of the oral flora and further can promote antibiotic resistance.

Improved efforts to treat and prevent periodontal disease which are simple and economical are sought.

### SUMMARY OF THE INVENTION

The invention is directed to devices and methods for preventing and inhibiting periodontal disease and in particular to devices and methods which mechanically and chemically disrupt biofilms formed by S. mutans.

According to one embodiment of the invention is a dental tool for preventing and inhibiting periodontal disease.

According to another embodiment of the invention is a dental tool for preventing and inhibiting periodontal disease by mechanically and chemically disrupting biofilms formed by S. mutans.

According to another embodiment of the invention is a dental tool for preventing and inhibiting periodontal disease by mechanically and chemically disrupting biofilms formed by S. mutans with a composition comprising a complex carbohydrate.

According to another embodiment of the invention is a dental tool for preventing and inhibiting periodontal disease by mechanically and chemically disrupting biofilms formed by S. mutans with a composition comprising a complex carbohydrate selected from β-glucans, α-glucans, fructans, galactans, xylans, glycosaminoglycans, pectins, chitin, chitosans, a cellulose and alginic acids.

Applicants have discovered that by mechanical and chemical disruption of a biofilm formed by S. mutans with a complex carbohydrate containing composition, the mechanism of biofilm formation can be inhibited, reducing the concentration of S. mutans and P. gingivalis in the oral cavity.

While not wishing to be bound by a specific theory, Applicants have discovered that biofilm formation from S. mutans can be inhibited by mechanically and chemically disrupting a biofilm with a complex carbohydrate agent which competitively binds to a cell surface expressed glucosyltransferase which inhibits binding of S. mutans to a biofilm, also decreasing biofilm formation. Reducing S. mutans concentration and inhibiting biofilm formation can reduce P. gingivalis concentration, preventing and inhibiting periodontal disease.

### DETAILED DESCRIPTION OF THE PREFERRED EMBODIMENTS

A complex carbohydrate containing compositions comprise a complex carbohydrate agent which in a preferred embodiment binds to a glucosyltransferase expressed on a cell surface of S. mutans. By binding to a surface expressed glucosyltransferase, binding of the S. mutans to a polyglucan biofilm may be inhibited, reducing the concentration of S. mutans and of biofilm.

Suitable complex carbohydrate agents are glucosyltransferase acceptors which competitively bind with glucosyltransferases relative to binding to biofilm glucan. Efficacy may be observed at concentrations of complex carbohydrate agents at 550 mMolar, preferably 400 mMolar, more preferably 300 mMolar, even more preferably 200 mMolar, even more preferably 100 mMolar, even more preferably 10 mMolar, even more preferably 1 mMolar, and as low as 10 μMolar.

Within the context of the present invention the term complex carbohydrate includes both oligosaccharides and polysaccharides.

Specific examples of complex carbohydrate agents are oligosaccharides which include: β-glucans, α-glucans, fructans, galactans, xylans, glycosaminoglycans, pectins, chitin, chitosans, a cellulose and alginic acids.

### β-Glucans

β-Glucans comprise polymers of β-D-linked glucose monomers, naturally occurring in cereals such as oat, barley and wheat β-glucans. Typically, β-glucans form a linear backbone with β-1,4 glycosidic bonds interspersed with occasional β-1,3 bonds.

β-glucans may be prepared by conventional methods known to those of ordinary skill in the art. Molecular weights from 360-100,000, preferably 900-50,000, more preferably 1800 to 20,000.

Specific non-limiting types of β-glucans are β-glucan in all MWs (mostly β-1,4); laminarin (β-1,3 and β-1,6); zymosan (β-1,2); and curdlan (β-1,3). The structure and preparation of such β-glucans are well known to those of ordinary skill in the art, without undue experimentation.

Oat β-glucans are water-soluble β-glucans derived from the endosperm of oat kernels known for their dietary contribution as components of soluble fiber. Cereal β-glucans—including β-glucan from oat, barley and wheat—are linear polysaccharides joined by 1,3 and 1,4 carbon linkages. The majority of cereal β-glucan bonds consist of 3 or 4 β-1,4 glycosidic bonds (trimers and tetramers)interconnected by 1,3 linkages. In β-glucan, these trimers and tetramers are known as cellotriosyl and cellotetraosyl. Oats and barley differ in the ratio of cellotriosyl to cellotetraosyl, and barley has more 1-4 linkages with a degree of polymerization higher than 4. In oats, β-glucan is found mainly in the endosperm of the oat kernel, especially in the outer layers of that endosperm (a marked difference from barley, which contains β-glucan uniformly throughout the endosperm). β-glucan extraction from oat can be difficult due to tendency of depolymerization—which often occurs in high pH. Thus β-glucan extraction is usually performed under a more neutral pH and generally at temperatures of 60-100° C. Usually β-glucan is solubilized in the extraction process with residual starch, which is then removed by hydrolysis with alpha-amylase. The residual solution usually contains coextracts of hemicelluloses and proteins which can then be separated through selective precipitation. Through wet milling, sieving, and solvent-extraction, oat beta-glucans can achieve up to 95% extraction purity.

### α-Glucans

α-Glucans include dextrans, amyloses and starches.

Dextran is a complex branched glucan derived from the condensation of glucose having predominantly 1,6 glycosidic bonds but may also have branches from α-1,3 linkages. Dextran may be made by conventional methods known to those of ordinary skill in the art, without undue experimentation, such as by fermentation of lactobacillus with sucrose. Molecular weights from 360-100,000, preferably 900-50,000, more preferably 1800 to 20,000.

### Fructans

Fructans are built up of fructose residues, normally with a sucrose unit (i.e. a glucose-fructose disaccharide) at what would otherwise be the reducing terminus. Linkage normally occurs at one of the two primary hydroxyls (OH-1 or OH-6), and there are two basic types of simple fructan: 1-linked: In inulin, the fructosyl residues are linked by β-2,1-linkages. In levan (or phlein), the fructosyl residues are linked by β-2,6-linkages. A third type of fructans, the graminan-type, contains both β-2,1-linkages and β-2,6-linkages. More complex fructans are formed on a 6G-kestotriose backbone where elongations occur on both sides of the molecule. Two types are discerned: neo-inulin type: pre-dominantly β-2,1-linkages; and neo-levan type: pre-dominantly β-2,6-linkages.

Fructans may be prepared by conventional methods known to those of ordinary skill in the art, without undue experimentation. Molecular weights from 360-100,000, preferably 900-50,000, more preferably 1800 to 20,000.

### Galactans

Galactans are complex carbohydrates of polymerized galactose. In general, galactans in natural sources contain a core of galactose units connected by α-1,3 or α-1-6 linkages, with structures containing other monosaccharides as side-chains.

Carrageenans are high-molecular-weight a complex carbohydrates made up of repeating galactose units and 3,6 anhydrogalactose (3,6-AG), both sulfated and nonsulfated. The units are joined by alternating α-1,3 and β-1,4 glycosidic linkages.

There are three main commercial classes of carrageenan:

Kappa forms strong, rigid gels in the presence of potassium ions, and reacts with dairy proteins. It is sourced mainly from Kappaphycus alvarezii.

Iota forms soft gels in the presence of calcium ions. It is produced mainly from Eucheuma denticulatum.

Lambda does not gel, and is used to thicken dairy products.

Galactans may be prepared by conventional methods known to those of ordinary skill in the art, without undue experimentation. Molecular weights from 360-100,000, preferably 900-50,000, more preferably 1800 to 20,000.

### Xylans

Xylans are o a complex carbohydrates made up of β-1,4-linked xylose residues with side branches of α-arabinofuranose and α-glucuronic acids and contribute to cross-linking of cellulose microfibrils and lignin through ferulic acid residues. On the basis of substituted groups, xylan can be categorized into three classes i) glucuronoxylan (GX) ii) neutral arabinoxylan (AX) and iii) glucuronoarabinoxylan (GAX).

Xylans may be prepared by conventional methods known to those of ordinary skill in the art, without undue experimentation. Molecular weights from 360-100,000, preferably 900-50,000, more preferably 1800 to 20,000.

### Glycosaminoglycans

Glycosaminoglycans are long unbranched a complex carbohydrates consisting of a repeating copolymer of an amino sugar (N-acetylglucosamine or N-acetylgalactosamine) along with a uronic acid (glucuronic acid or iduronic acid) or galactose. Glycosaminoglycans may be classified into four groups: Heparin/heparan sulfate (HSGAGs); chondroitin sulfate/dermatan sulfate (CSGAGs); keratan sulfate; and hyaluronic acid.

Glycosaminoglycans may be prepared by conventional methods known to those of ordinary skill in the art, without undue experimentation. Molecular weights from 360-100,000, preferably 900-50,000, more preferably 1800 to 20,000.

### Pectin

Pectins are rich in galacturonic acid. Several distinct a complex carbohydrates have been identified and characterized within the pectic group. Homogalacturonans are linear chains of α-(1-4)-linked D-galacturonic acid. Substituted galacturonans are characterized by the presence of saccharide appendant residues (such as D-xylose or D-apiose in the respective cases of xylogalacturonan and apiogalacturonan) branching from a backbone of D-galacturonic acid residues. Rhamnogalacturonan I pectins (RG-I) contain a backbone of the repeating disaccharide: 4)-α-D-galacturonic acid-(1,2)-α-L-rhamnose-(1. From many of the rhamnose residues, sidechains of various neutral sugars branch off. The neutral sugars are mainly D-galactose, L-arabinose and D-xylose, with the types and proportions of neutral sugars varying with the origin of pectin.

Another structural type of pectin is rhamnogalacturonan II (RG-II), which is a less frequent, complex, highly branched a complex carbohydrate. Rhamnogalacturonan II is classified by some authors within the group of substituted galacturonans since the rhamnogalacturonan II backbone is made exclusively of D-galacturonic acid units.

Isolated pectin has a molecular weight of typically 60,000-130,000 g/mol, varying with origin and extraction conditions.

The non-esterified galacturonic acid units can be either free acids (carboxyl groups) or salts with sodium, potassium, or calcium. The salts of partially esterified pectins are called pectinates, if the degree of esterification is below 5 percent the salts are called pectates, the insoluble acid form, pectic acid.

Pectins may be prepared by conventional methods known to those of ordinary skill in the art, without undue experimentation.

### Chitin

Chitin is a long-chain polymer of N-acetylglucosamine. The structure of the chitin molecule is of two of the N-acetylglucosamine units that repeat to form long chains in β-1,4-linkage.

Chitin may be prepared by conventional methods known to those of ordinary skill in the art. Molecular weights from 360-4,000, preferably 540-3,000, more preferably 720 to 1,800.

### Chitosan

Chitosan is a linear a complex carbohydrate composed of randomly distributed β-1,4-linked D-glucosamine (deacetylated unit) and N-acetyl-D-glucosamine (acetylated unit).

Chitosans may be prepared by conventional methods known to those of ordinary skill in the art such as by treating chitin with an alkaline substance, like sodium hydroxide.

Molecular weights from 360-4,000, preferably 540-3,000, more preferably 720 to 1,800.

### Cellulose

Cellulose is an organic compound with the formula (C₆H₁₀O₅)ₙ, a polysaccharide consisting of a linear chain of several hundred to many thousands of β-1,4 linked D-glucose units. Cellulose is an important structural component of the primary cell wall of green plants, many forms of algae and the oomycetes. Some species of bacteria secrete it to form biofilms.

Celluloses may be prepared by conventional methods known to those of ordinary skill in the art.

Specific non-limiting types of cellulose are low molecular-weight celluloses (all β-1,4) and in particular cellulose trisaccharide (cellotriose), hydroxymethylcellulose, hydroxyethylcellulose and carboxymethycellulose.

Molecular weights from 360-4,000, preferably 540-3,000, more preferably 720 to 1,800.

### Alginic Acids

Alginic acid is a linear copolymer with homopolymeric blocks of β-1,4-linked-D-mannuronate (M) and its C-5 epimer α-L-guluronate (G) residues, respectively, covalently linked together in different sequences or blocks. The monomers can appear in homopolymeric blocks of consecutive G-residues (G-blocks), consecutive M-residues (M-blocks) or alternating M and G-residues (MG-blocks).

Alginic acids may be prepared by conventional methods known to those of ordinary skill in the art such as from brown seaweeds by the: 1) Calcium alginate method or, 2) Alginic acid method. Molecular weights from 360-100,000, preferably 900-50,000, more preferably 1,800 to 20,000.

The complex carbohydrate agent is preferably water soluble, demonstrating a water solubility of at least 10 mM in water at 23° C., preferably at least 20 mM at 23° C., more preferably at least 50 mM at 23° C., even more preferably more preferably at least 100 mM at 23° C., even more preferably 1 M at 23° C., even more preferably 10 M at 23° C.

Specific solubilities are: dextran, 50 mg/ml; hydroxymethylcellulose, 10 mg/ml; cellulose, 0 mg/ml; oat beta glucan, 20 mg/ml.

### Complex Carbohydrate Containing Compositions

Compositions may be formulated as a liquid, a suspension, an emulsion, or a powder.

The concentration of complex carbohydrate in the complex carbohydrate containing composition is sufficient to effectively inhibit formation of a biofilm formed by S. mutans. Exemplary concentrations are at least 10 mM in water at 23° C., preferably at least 20 mM at 23° C., more preferably at least 50 mM at 23° C., even more preferably more preferably at least 100 mM at 23° C.

Liquid compositions may comprise complex carbohydrate agent and an orally acceptable solvent such as water and or ethanol and may further comprise additives such as glycerin, sorbitol and propylene glycol.

Thickening agents known to those of ordinary skill in the art may be added to increase viscosity.

Suspensions of complex carbohydrate agents may be prepared by conventional methods known to those of ordinary skill in the art without undue experimentation using orally acceptable liquids known in the field of dental care. A suspension aid may also be used.

The complex carbohydrate composition may be in the form of a water-in-oil emulsion or microemulsion. The complex carbohydrate would be dissolved in an aqueous phase of the water-in-oil emulsion or microemulsion in the form of vesicles stabilized by surfactants. The formation of a water-in-oil emulsion and microemulsion is understood by those of ordinary skill in the art without undue experimentation.

The complex carbohydrate composition may also be microencapsulated with coatings such as ethyl cellulose, polyvinyl alcohol, gelatin and sodium alginate. The coating thickness would be sufficient to allow fracture of the microcapsule during mechanical disruption of a biofilm. Suitable microencapsulation techniques are known to those of ordinary skill in the art, without undue experimentation.

The complex carbohydrate containing composition may be in the form of a powder having a D₅₀ 500-1,000 μm, preferably 600-900 μm., more preferably 700-800 μm.

The pH of such compositions described herein is generally in the range of from about 5 to about 9 and typically from about 5 to about 7. The solubility of an oligosaccharide in the composition may be adjusted by adjusting the pH. In particular uronic acid containing oligosaccharides may be better solubilized at a basic pH. The pH can be controlled with acid (e.g. citric acid or benzoic acid) or base (e.g. sodium hydroxide) or buffered (as with sodium citrate, benzoate, carbonate, or bicarbonate, disodium hydrogen phosphate, sodium dihydrogen phosphate, etc.).

Solubilizing agents may also be included such as humectant polyols such propylene glycol, dipropylene glycol and hexylene glycol, cellosolves such as methyl cellosolve and ethyl cellosolve, vegetable oils and waxes containing at least about 12 carbons in a straight chain such as olive oil, castor oil and petrolatum and esters such as amyl acetate, ethyl acetate and benzyl benzoate.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added. These compositions may be preserved by the addition of an antioxidant such as ascorbic acid.

### Dental Tool

The complex carbohydrate composition may be delivered to a biofilm surface by many techniques known to those of ordinary skill in the art when applied to a surface of or incorporated into a dental tool such as, a floss/tape, an interdental brush, a tooth brush, a pick, a chewable tablet or a chewing gum. The complex carbohydrate composition may be applied to a surface of a delivery vehicle in the form of a solution, emulsion, microemulsion, microcapsule or powder. The complex carbohydrate composition may be incorporated into a delivery vehicle such as a chewable tablet or chewing gum in the form of a solution, an aqueous solution, an emulsion, a microemulsion, a microcapsule or a powder.

### Floss

Flossing substrate is substantially planar in construction and may be made of expanded polytetrafluoroethylene (ePTFE). In one embodiment, the substrate may be multi-filament nylon or non-elastic ultra-high molecular weight polyethylene (UHMWPE) or the like. Of course those skilled in the art will realize that other materials may be employed for the substrate and new materials adapted for such may come available. As such, any materials which one skilled in the art might employ for the substrate are considered within the scope of this application.

The floss may be in the form of a single ribbon (e.g., a Teflon® or polyethylene ribbon). Alternatively, it may be bundle of thin filaments, e.g., nylon filaments. The number of filaments will be from about 2 to about 300, e.g., from about 2 to about 200, depending on the denier of the filaments. The filaments are twisted with about 1 to 5 twists per inch to form the floss. The twisting provides integrity of the floss on the spool and during subsequent handling. However, when used, the filaments will spread out and splay against tooth surfaces. The floss may also be formed of interlocking fibers, e.g., as in the case Oral-B Ultra Floss™. In any case the final floss product is preferably of a thickness that allows it to fit between the teeth. The floss may be coated with a wax. Where multiple filaments are used, the coating may be applied before or after twisting, preferably after twisting. Other additives may be applied to a wax coated floss after the wax coating. The flavor can be applied as a liquid or a solid. It is preferred to use a spray dried solid. Likewise, the various other additives can be applied as a liquid or a solid. When applied as a liquid the floss is dried prior to being wound onto a spool. The drying can be by radiant drying or air drying. After drying, the floss is wound onto a spool.

Methods of manufacturing dental floss are well known in the art. For example, dental floss may be produced from nylon, as nylon salt is polymerized, and the resulting polymer is pumped or extruded to form monofilaments. The filaments are allowed to harden, and then combined to form a strand of floss. Dental floss may be produced from polytetrafluoroethylene (PTFE or Teflon®), polypropylene, polyethylene, styrene butadiene copolymers, combination of them. The polymer is melted and extruded into thin strands. See also U.S. Pat. No. 6,270,890.

In one embodiment, resin, e.g., nylon or PTFE, is mixed with an oligosaccharide, or a salt thereof, and extruded to form a filament (e.g., in the case of nylon) which are twisted to form the floss, or formed into a single ribbon of floss (e.g., in the case of PTFE). It should be understood that some of complex carbohydrate or salt will be disposed near the surface of the floss, and will be exposed and released when the dental floss is used. In one embodiment, the floss has a denier of about 450 to about 1350. In another embodiment the dernier of the floss is from about 100 to about 900.

Methods for coating dental floss are known in the art. In one embodiment of the present invention, the dental floss is treated in an emulsion bath comprising complex carbohydrate or a pharmaceutically acceptable salt thereof. The emulsion bath may optionally contain one or more waxes, which adhere to the floss, and thereby cause the complex carbohydrate to adhere to the floss. In another embodiment, a dental floss comprising a non-PTFE fiber is coated with a first and a second coating overlaying the first coating. The first coating is a nylon bonding coating, and the second coating is a wax or polymer, e.g., such as polyvinyl alcohol, polyvinyl acetate, etc., in combination or association with an oligosaccharide or salt thereof. See e.g., U.S. Pat. No. 6,289,904.

In one embodiment of the present invention, the dental floss may optionally include fluoride, or a fluoride ion source. A wide variety of fluoride ion-yielding materials can be employed as sources of soluble fluoride in the present compositions. Examples of suitable fluoride ion-yielding materials are found in Briner et al. U.S. Pat. No. 3,535,421; Parran, Jr. et al. U.S. Pat. No. 4,885,155, and Widder et al. U.S. Pat. No. 3,678,154. Representative fluoride ion sources include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes stannous fluoride, sodium fluoride, sodium monofluorophosphate as well as mixtures thereof.

The dental floss of the present invention may also comprise abrasive particles, e.g., aluminum oxide, small particle silica, or other abrasive or polishing particles. See e.g., U.S. Pat. No. 6,453,912.

The dental floss of the present invention may also comprise an antiseptic or antimicrobial selected from triclosan, herbal extracts and essential oils (e.g. rosemary extract, thymol, menthol, eucalyptol, methyl salicylate), bisguanide antiseptics (e.g., chlorhexidine, alexidine or octenidine), quaternary ammonium compounds (e.g., cetylpyridinium chloride), phenolic antiseptics, hexetidine, povidone iodine, delmopinol, salifluor, metal ions (e.g., zinc salts, for example, zinc citrate), sanguinarine, propolis, and combinations thereof to further aid in the beneficial effects of the complex carbohydrate.

As use of dental floss may cause discomfort or bleeding during or after use, it may optionally comprise analgesic agents, anti-inflammatory agents, coagulants, vitamins, and combinations thereof.

Dental floss as described above may also be used incorporated into a dental floss wand, dental tape or a floss pick.

The complex carbohydrate containing compositions may also be delivered to the surface of a biofilm, as applied to a surface of or incorporated into the structure of a dental tool such as to bristles of a toothbrush, bristles of an interdental brush or the structure of a toothpick. Under the mechanical action of the dental tool, the structure of the biofilm is disrupted, exposing an area of the biofilm, below the surface to mechanical and chemical action of a complex carbohydrate agent. In one embodiment a complex carbohydrate containing composition is applied to a surface of a dental tool and dried before use. In another embodiment, a liquid complex carbohydrate containing composition is applied to a surface of a dental tool and used without drying. In another embodiment, a powder or microcapsules of complex carbohydrate containing composition is adhered to a surface of a dental tool.

The complex carbohydrate containing composition may be incorporated into an oral delivery vehicle such as a chewable tablet or a chewing gum. The formation of a chewable tablet and/or a chewing gum containing a complex carbohydrate containing composition is well known to those of ordinary skill in the art, without undue experimentation. A chewable tablet may find particular application in veterinary oral care.

### Floss/Tape Dispenser

Dental floss is commonly supplied in plastic dispensers that contain 10 to 50 meters of floss. The dispenser typically has a small protected blade used to sever the floss when a desired amount is pulled out.

In one embodiment, of the present invention, a dental floss dispenser is provided which contains a complex carbohydrate or salt thereof, in the form of solution, an emulsion, a microemulsion or a powder thereof is disposed within the container and in contact with the floss. As the floss is stored or as a user pulls out a desired amount of floss, the floss comes in contact with the complex carbohydrate, salt, or solution, thereby coating the floss.

### Method of Preventing and/or Inhibiting Periodontal Disease

In another embodiment, a method of preventing and/or inhibiting periodontal disease is described by mechanically and chemically disrupting a biofilm with a dental tool comprising a complex carbohydrate containing composition.

Biofilms produced by S. mutans have a structure comprising poly β-glucans, synthesized from sucrose under the action of glucosyltransferases. Once established, the glucan rich biofilm provides a matrix for oral bacteria such as S. mutans and P. gingivalis.

Under the mechanical action of a dental tool comprising a complex carbohydrate agent, the biofilm structure may be disrupted, exposing S. mutans contained therein wherein the complex carbohydrate agent may act as a glucosyltransferase acceptor, binding with a S. mutans cell surface bound glucosyltransferase. Accordingly under the mechanical action of a dental tool comprising a complex carbohydrate agent, 1) biofilm is physically disrupted which can facilitate reduction and removal of biofilm mass; and 2) biofilm formation may be reduced and/or suppressed by reducing the adherence of S. mutans and thus the concentration of S. mutans associated with the biofilm and by inhibiting the synthesis of biofilm by inhibiting the action of glucosyltransferase on sucrose in the synthesis of β-glucan. Such a mechanical and chemical action on oral biofilm reduces a matrix for oral P. gingivalis an active agent for periodontal disease and thus prevents and/or inhibits periodontal disease.

While there is no functional upper limit to the amount of complex carbohydrate agent delivered to the biofilm, the method delivers a glucosyltransferase inhibiting effective amount of complex carbohydrate agent, such as a concentration of at the biofilm of at least 10 mM, preferably at least 20 mM, more preferably at least 50 mM, even more preferably more preferably at least 100 mM.

Mechanical and chemical disruption of a biofilm with a dental tool comprising a complex carbohydrate agent can achieve glucan synthesis inhibition of at least 10%, preferably at least 20%, more preferably at least 30%, even more preferably at least 40%, even more preferably at least 50%, even more preferably at least 60%, even more preferably at least 70%, relative to undisrupted biofilm formation.

Use of a dental tool comprising a complex carbohydrate agent to disrupt a biofilm would be analogous to dental tool use in the absence of a complex carbohydrate agent and may be by techniques practiced by those of ordinary skill in the art of personal dental hygiene, without undue experimentation.

### Patients

The complex carbohydrate containing composition is envisioned as suitable for oral application to mammals which are subject to periodontal disease and which harbor S. mutans. The patient can be a human, canines, equine, bovine or felines.

In a canine embodiment the dental tool may specifically be a tablet or a chew comprising a complex carbohydrate containing composition. A complex carbohydrate containing composition may be incorporated into canine dental treats and canine chew toys, known to those of ordinary skill in the art of veterinary art.

Also in the canine embodiment, brushes comprising a complex carbohydrate agent and canine toothpastes comprising an oligosaccharide agent may be directly applied to canine teeth.

Obviously, numerous modifications and variations of the present invention are possible in light of the above teachings. It is therefore to be understood that within the scope of the appended claims, the invention may be practiced otherwise than as specifically described herein.

1. A dental tool comprising:
a substrate; and
a composition comprising a complex carbohydrate,
wherein said dental tool is selected from the group consisting of a floss, a tape, a floss pick, an interdental brush, and a pick, and
wherein said complex carbohydrate is a β-glucan having a molecular weight of said β-glucan is 360 to 20,000.

2. (canceled)

3. (canceled)

4. (canceled)

5. The dental tool according to claim 14, wherein said β-glucan is at least one selected from the group consisting of oat glucan, cellulose, hydroxymethylcellulose, hydroxyethylcellulose and carboxymethylcellulose.

6. The dental tool according to claim 1, wherein said tool is a dental floss.

7. The dental tool according to claim 6, wherein said substrate is a polymer, wherein said polymer is coated or impregnated with said composition.

8. The dental tool according to claim 6, wherein said dental floss has a denier of 100 to 1350.

9. (canceled)

10. The dental tool according to claim 1, wherein said complex carbohydrate has a solubility in water of at least 10 mM in water at 23° C.

11. A method of preventing and/or inhibiting periodontal disease comprising mechanically and chemically disrupting a biofilm with a dental tool comprising a complex carbohydrate composition,
wherein said dental tool is selected from the group consisting of a floss, a tape, a floss pick, an interdental brush, and a pick, and
wherein said complex carbohydrate is a β-glucan having a molecular weight of said β-glucan is 360 to 20,000.

12. The method according to claim 11, wherein said biofilm is located within an oral cavity of a mammal.

13. The method according to claim 12, wherein said mammal is a selected from the group consisting of human, canines, equine, bovine and felines.

14. (canceled)

15. (canceled)

16. (canceled)

17. The method according to claim 11, wherein said β-glucan is at least one selected from the group consisting of oat glucan, cellulose, hydroxymethylcellulose, hydroxyethylcellulose and carboxymethylcellulose.

18. The method according to claim 11, wherein said complex carbohydrate has a solubility in water of at least 10 mM in water at 23° C.

19. (canceled)

20. (canceled)

21. (canceled)

22. (canceled)

23. The dental tool according to claim 1, wherein said complex carbohydrate bonds to a glycosyltransferase expressed on a cell surface of S. mutans.

24. The dental tool according to claim 1, wherein said β-glucan is an oat glucan.

25. The dental tool according to claim 1, wherein said β-glucan is cellulose.

26. The dental tool according to claim 1, wherein said β-glucan is hydroxymethylcellulose.

27. The dental tool according to claim 1, wherein said β-glucan is hydroxyethylcellulose.

28. The dental tool according to claim 1, wherein said β-glucan is carboxymethylcellulose.

29. The method according to claim 11, wherein said β-glucan is an oat glucan.

30. The method according to claim 11, wherein said β-glucan is cellulose.

31. The method according to claim 11, wherein said β-glucan is hydroxymethylcellulose.

32. The method according to claim 11, wherein said β-glucan is hydroxyethylcellulose.

33. The method according to claim 11, wherein said β-glucan is carboxymethylcellulose.
